SEQUENCE LISTING (4 sequences)

SEQ ID NO: 1 - DNA, 1380 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 2 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 3 - DNA, 2685 bp, Saccharomycopsis fibuligera
atggtctccttcacctccctcctcgccggcgtcgccgccatctcgggcgtcttggccgctcccgccgccgaggtcgaatccgtggctgtggagaagcgctcgcgagtcccaattcaaaactatacccagtctccatcccagagagatgagagctcccaatgggtgagcccgcattattatccaactccacaaggtggtaggctccaagacgtctggcaagaagcatatgctagagcaaaagccatcgttggccagatgactattgttgaaaaggtcaatttgaccactggtaccggttggcaattagatccatgtgttggtaataccggttctgttccaagattcggcatcccaaacctttgcctacaagatgggccattgggtgttcgattcgctgactttgttactggctatccatccggtcttgctactggtgcaacgttcaataaggatttgtttcttcaaagaggtcaagctctcggtcatgagttcaacagcaaaggtgtacatattgcgttgggccctgctgttggcccacttggtgtcaaagccagaggtggcagaaatttcgaagcctttggttccgacccatatctccaaggtactgctgctgctgcaaccatcaaaggtctccaagagaataatgttatggcttgtgtcaagcactttattggtaacgaacaagaaaagtacagacagccagatgacataaaccctgccaccaaccaaactactaaagaagctattagtgccaacattccagacagagccatgcatgcgttgtacttgtggccatttgccgattcggttcgagcaggtgttggttctgttatgtgctcttataacagagtcaacaacacttacgcttgcgaaaactcttacatgatgaaccacttgcttaaagaagagttgggttttcaaggctttgttgtttcggactggggtgcacaattaagtggggtttatagcgctatctcgggcttagatatgtctatgcctggtgaagtgtatgggggatggaacaccggcacgtctttctggggtcaaaacttgacgaaagctatttacaatgagactgttccgattgaaagattagatgatatggcaaccaggatcttggctgctttgtatgctaccaatagtttcccaacagaagatcaccttccaaatttttcttcatggacaacgaaagaatatggcaataaatattatgctgacaacactaccgagattgtcaaagtcaactacaatgtggacccatcaaatgactttacggaggacacagctttgaaggttgctgaggaatctattgtgcttttaaaaaatgaaaacaacactttgccaatttctcccgaaaaggctaaaagattactattgtcgggtattgctgcaggccctgatccgataggttatcagtgtgaagatcaatcttgcacaaatggcgctttgtttcaaggttggggttctggcagtgttggttctccaaaatatcaagtcactccatttgaggaaatttcttatcttgcaagaaaaaacaagatgcaatttgattatattcgggagtcttacgacttagctcaagttactaaagtagcttccgatgctcatttgtctatagttgttgtctctgctgcaagcggtgagggttatataaccgttgacggtaaccaaggtgacagaaaaaatctcactttgtggaacaacggtgataaattgattgaaacagttgctgaaaactgtgccaatactgttgttgttgttacttctactggtcaaattaattttgaaggctttgctgatcacccaaatgttaccgcaattgtctgggccggcccattaggtgacagatccgggactgctatcgccaatattctttttggtaaagcgaacccatcaggtcatcttccattcactattgctaagactgacgatgattacattccaattgaaacctacagtccatcgagtggtgaacctgaagacaaccacttggttgaaaatgacttgcttgttgactatagatattttgaagagaagaatattgagccaagatacgcatttggttatggcttgtcttacaatgagtatgaagttagcaatgcaaaggtctcggcagccaaaaaagttgatgaggagttgcctgaaccagctacctacttatcggagtttagctatcaaaatgcaaaagacagcaaaaatccaagtgatgcttttgctccagcagatttaaacagagttaatgagtacctttatccatatttagatagcaatgttaccttaaaagacggaaactatgagtatcctgatggctacagcactgagcaaagaacaacacctaaccaacctgggggcggcttgggaggcaacgatgctttgtgggaggtcgcttataactccactgataagtttgttccacagggtaactccactgataagtttgttccacagttgtatttgaaacaccctgaggatggcaagtttgaaacccctattcaattgagagggtttgaaaaggttgagttgtccccgggtgagaagaagacagttgatttgaggcttttgagaagagatcttagtgtgtgggataccaccagacagtcttggatcgttgaatctggtacttatgaggccttaattggcgttgctgttaatgatatcaagacatctgtcctgtttactatttga

SEQ ID NO: 4 - protein, 894 aa, Saccharomycopsis fibuligera
MVSFTSLLAGVAAISGVLAAPAAEVESVAVEKRSRVPIQNYTQSPSQRDESSQWVSPHYYPTPQGGRLQDVWQEAYARAKAIVGQMTIVEKVNLTTGTGWQLDPCVGNTGSVPRFGIPNLCLQDGPLGVRFADFVTGYPSGLATGATFNKDLFLQRGQALGHEFNSKGVHIALGPAVGPLGVKARGGRNFEAFGSDPYLQGTAAAATIKGLQENNVMACVKHFIGNEQEKYRQPDDINPATNQTTKEAISANIPDRAMHALYLWPFADSVRAGVGSVMCSYNRVNNTYACENSYMMNHLLKEELGFQGFVVSDWGAQLSGVYSAISGLDMSMPGEVYGGWNTGTSFWGQNLTKAIYNETVPIERLDDMATRILAALYATNSFPTEDHLPNFSSWTTKEYGNKYYADNTTEIVKVNYNVDPSNDFTEDTALKVAEESIVLLKNENNTLPISPEKAKRLLLSGIAAGPDPIGYQCEDQSCTNGALFQGWGSGSVGSPKYQVTPFEEISYLARKNKMQFDYIRESYDLAQVTKVASDAHLSIVVVSAASGEGYITVDGNQGDRKNLTLWNNGDKLIETVAENCANTVVVVTSTGQINFEGFADHPNVTAIVWAGPLGDRSGTAIANILFGKANPSGHLPFTIAKTDDDYIPIETYSPSSGEPEDNHLVENDLLVDYRYFEEKNIEPRYAFGYGLSYNEYEVSNAKVSAAKKVDEELPEPATYLSEFSYQNAKDSKNPSDAFAPADLNRVNEYLYPYLDSNVTLKDGNYEYPDGYSTEQRTTPNQPGGGLGGNDALWEVAYNSTDKFVPQGNSTDKFVPQLYLKHPEDGKFETPIQLRGFEKVELSPGEKKTVDLRLLRRDLSVWDTTRQSWIVESGTYEALIGVAVNDIKTSVLFTI

(19) United States
(12) Patent Application Publication
(10) Pub. No.: US 2011/0129888 A1
(43) Pub. Date: Jun. 2, 2011

(54) Method for Fermenting Cellulosics

(75) Inventors: Willem Heber Van Zyl, Stellenbosch (ZA); Riaan Den Haan, Durbanville (ZA)

(73) Assignee: Stellenbosch University, Stellenbosch (ZA)

(21) Appl. No.: 12/677,956

(22) PCT Filed: Sep. 14, 2007
(86) PCT No.: PCT/IB2007/004098

§ 371 Date: Oct. 25, 2010

(51) Int. Cl. C12P 7/10; C12N 1/19

(52) U.S. Cl. 435/165; 435/254.2; 435/254.21; 435/254.22; 435/254.23

(57) ABSTRACT

The present invention is directed to host cells capable of fermenting cellulosic materials for the production of ethanol. Microorganisms engineered to be able to use amorphous cellulosic materials in a fermentation process to produce ethanol are disclosed. Additionally, methods of using the host organisms of the invention and compositions for producing ethanol according to the invention are disclosed.

### BACKGROUND OF THE INVENTION

1. Field of the Invention

The present invention relates to the field of metabolic engineering. Particularly, the invention relates to engineering organisms to ferment biomass material for the production of ethanol.

2. Background Art

On a world-wide base, 1.3×10¹⁰ metric tons (dry weight) of terrestrial plants are produced annually (Demain, A. L., et al., Microbiol. Mol. Biol. Rev. 69, 124-154 (2005)). Plant biomass consists of about 40-55% cellulose, 25-50% hemicellulose and 10-40% lignin, depending whether the source is hardwood, softwood, or grasses (Sun, Y. and Cheng, J., Bioresource Technol. 83, 1-11 (2002)). The major polysaccharide present is water-insoluble cellulose that contains the major fraction of fermentable sugars (glucose, cellobiose or cellodextrins). Native cellulose consists of amorphous and crystalline regions (FIG. 1).

Three major types of enzymatic activities are required for native cellulose degradation: (1) endoglucanases (1,4-β-D-glucan 4-glucanohydrolases; EC 3.2.1.4), (2) exoglucanases, including cellodextrinases (1,4-β-D-glucan glucanohydrolases; EC 3.2.1.74) and cellobiohydrolases (1,4-β-D-glucan cellobiohydrolases; EC 3.2.1.91); and (3) β-glucosidases (β-glucoside glucohydrolases; EC 3.2.1.21). Endoglucanases cut at random in the cellulose polysaccharide chain of amorphous cellulose, generating oligosaccharides of varying lengths and consequently new chain ends. Exoglucanases act in a processive manner on the reducing or non-reducing ends of cellulose polysaccharide chains, liberating either glucose (glucanohydrolases) or cellobiose (cellobiohydrolase) as major products. Exoglucanases can also act on microcrystalline cellulose, presumably peeling cellulose chains from the microcrystalline structure. β-Glucosidases hydrolyze soluble cellodextrins and cellobiose to glucose units (FIG. 1).

A variety of plant biomass resources are available as lignocellulosics for the production of biofuels, notably bioethanol. The major sources are (i) wood residues from paper mills, sawmills and furniture manufacturing, (ii) municipal solid wastes, (iii) agricultural residues and (iv) energy crops. Pre-conversion of particularly the cellulosic fraction in these biomass resources (using either physical, chemical or enzymatic processes) to fermentable sugars (glucose, cellobiose and cellodextrins) would enable their fermentation to bioethanol, provided the necessary fermentative micro-organism with the ability to utilize these sugars is used.

Bakers' yeast (Saccharomyces cerevisiae) remains the preferred micro-organism for the production of ethanol (Hahn-Hagerdal, B., et al., Adv. Biochem. Eng. Biotechnol. 73, 53-84 (2001)). Attributes in favor of this microbe are (i) high productivity at close to theoretical yields (0.51 g ethanol produced/g glucose used), (ii) high osmo- and ethanol tolerance, (iii) natural robustness in industrial processes, and (iv) being generally regarded as safe due to its long association with wine and bread making, and beer brewing. The major shortcoming of S. cerevisiae is its inability to utilize complex polysaccharides such as cellulose, or its break-down products, such as cellobiose and cellodextrins.

With the aid of recombinant DNA technology, cellulases from bacterial and fungal sources have been transferred to S. cerevisiae, enabling the degradation of cellulosic derivatives (Van Rensburg, P., et al., Yeast 14, 67-76 (1998)), or growth on cellobiose (Van Rooyen, R., et al., J. Biotech. 120, 284-295 (2005)); McBride, J. E., et al., Enzyme Microb. Techol. 37, 93-101 (2005)).

Fujita, Y., et al., (Appl. Environ. Microbiol. 70, 1207-1212 (2004)) attempted to extend this previous work and engineer yeast to ferment cellulosic material by immobilizing various enzymes on the yeast surface. However, Fujita et al. were unable to achieve fermentation of amorphous cellulose using yeast expressing only recombinant endoglucanase and β-glucosidase. Another limitation of the Fujita et al. approach was that cells had to be pre-grown to high cell density on standard carbon sources before the cells were useful for ethanol production using amorphous cellulose. For example, Fujita et al. teach high biomass loadings of ˜15 g/L to accomplish ethanol production.

### BRIEF SUMMARY OF THE INVENTION

In one aspect of the present invention, recombinant host cells are able to grow and multiply on amorphous cellulose and concomitantly produce ethanol. This improvement brings about drastic cost savings and increased industrial efficiency by not requiring extensive pre-growth of the cells.

Another aspect of the invention relates to a recombinant host cell comprising a heterologous polynucleotide which encodes an endoglucanase and a heterologous polynucleotide which encodes a β-glucosidase, wherein the host cell does not express an exoglucanase, and wherein the host cell can grow on amorphous cellulose as the sole carbon source.

Another aspect of the invention relates to a recombinant host cell comprising a heterologous polynucleotide which encodes an endoglucanase and a heterologous polynucleotide which encodes a β-glucosidase, wherein the host cell can grow on amorphous cellulose as the sole carbon source, and wherein the host cell does not require pre-growth on a non-amorphous cellulose carbon source.

A further aspect of the present invention relates to a recombinant host cell comprising a heterologous polynucleotide which encodes an endoglucanase and a heterologous polynucleotide which encodes a β-glucosidase that can grow on amorphous cellulose as the sole carbon source and concomitantly produce ethanol.

Yet another aspect of the invention relates to methods for direct fermentation of amorphous cellulosics to ethanol, utilizing a recombinant yeast strain producing both endoglucanase and β-glucosidase enzymes.

In certain embodiments of the invention, methods of producing ethanol are disclosed that comprise contacting a composition comprising amorphous cellulose with a recombinant host cell wherein the host cell ferments amorphous cellulose to ethanol and then recovering the ethanol.

Other embodiments of the invention relate to compositions comprising recombinant host cells capable of fermenting cellulosic material.

### BRIEF DESCRIPTION OF FIGURES

FIG. 1 depicts a schematic representation of the hydrolysis of amorphous and microcrystalline cellulose by noncomplexed cellulases. The solid circles represent reducing ends, and the open circles represent nonreducing ends. Amorphous and crystalline regions are indicated. Cellulose, enzymes, and hydrolytic products are not shown to scale.

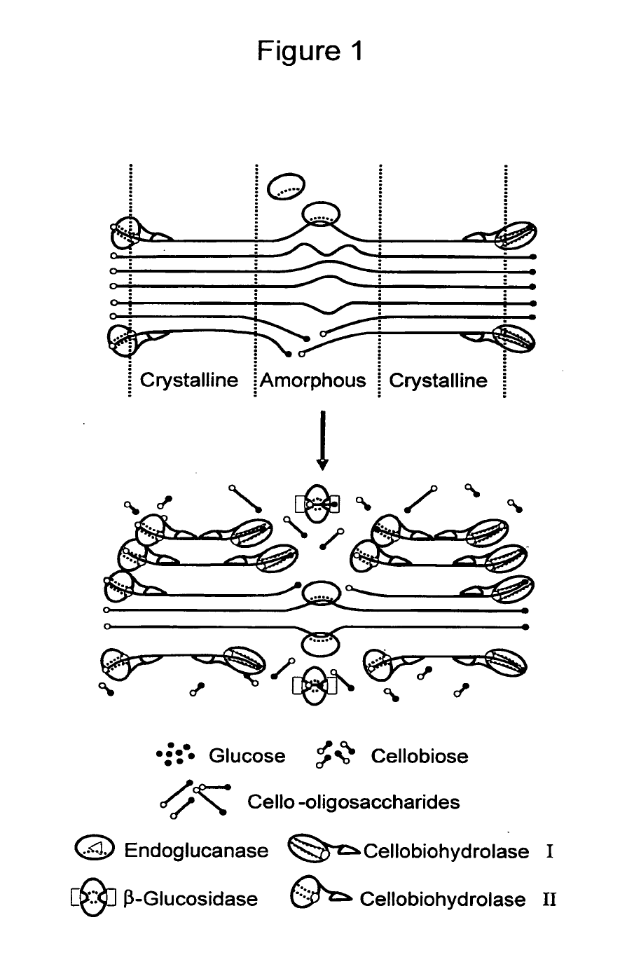

FIG. 2 depicts a schematic representation of the pCEL5 plasmid constructed in the Example. The 2μ sequence is responsible for episomal replication of the plasmid, and the S. cerevisiae orotidine-5′-phosphate decarboxylase (URA3) is used as a selectable marker. ENO1_{P}, ENO1_{T}, PGK1_{P}, PGK1_{T}, represents the S. cerevisiae enolase 1 and phosphoglycerate kinase 1 promoter and terminator DNA sequences; T. reesei EGI represents the endoglucanase I gene of Trichoderma reesei, XYNseq the 23-amino acid secretion signal of the xylanase II gene of T. reesei and S. fibuligera BGLI the β-glucosidase I gene of Saccharomycopsis fibuligera.

FIG. 3 depicts recombinant S. cerevisiae Y294 strains as plate cultures. (A) SC^{-URA} medium with 20 g·L⁻¹ glucose. (B) YPC medium (10 g·L⁻¹ cellobiose) showing growth of BGL1 containing Y294 strains. (C) SC^{-URA} medium (20 g·L⁻¹ glucose) supplemented with 0.1% CMC; after incubation colonies were washed and the medium was stained with Congo red. CMC degrading Y294 strains (containing EG1) show clearing zones. (D) YP-PASC (10 g·L⁻¹ PASC) medium showing growth of the BGL1, EG1 co-expressing strain Y294[CEL5]. (E) An enhanced topview of the YP-PASC plate in D to illustrate growth by strain Y294[CEL5]. The plates were photographed after 4 days of incubation at 30° C.

FIG. 4 depicts a time course of enzymatic activity of recombinant S. cerevisiae strains (as indicated) on YPD medium. (A) β-Glucosidase activity, indicated as total activity (supernatant and cell associated—solid symbols) and extracellular activity (supernatant)—open symbols was measured on p-NPG. (B) Extracellular endoglucanase activity was measured on CMC. Symbols used were Y294[Ref] (▾, ∇); Y294[SFI] (▴, Δ); Y294[EGI] (♦, ⋄); Y294[CEL5] (, ◯).

FIG. 5 depicts (A) growth curve (solid symbols) and (B) ethanol production (open symbols) time course of anaerobic cultures of recombinant S. cerevisiae strains (as indicated), on YP medium containing 10 g·L⁻¹ PASC as sole carbohydrate source. Symbols used were Y294[Ref] (▾, ∇); Y294[SFI] (▴, Δ); Y294[EGI] (♦, ⋄); Y294[CEL5] glucose preculture (, ◯); Y294[CEL5] PASC preculture (▪, □).

FIG. 6 depicts decreased viscosity of anaerobic YP-PASC cultures at the end of the 240 hour growth period. Viscosity measurements were done over 30 shear rates (2-200 s⁻¹) for the culture media after the growth period as well as for fresh YP-PASC (10 g·L⁻¹ PASC) medium. The average viscosities of the spent culture media were expressed as a percentage of the viscosity of fresh medium.

FIG. 7 depicts a growth curve (solid symbols) of aerobic cultures of recombinant S. cerevisiae strains (as indicated), on YP medium containing 10 PASC as the sole carbohydrate source. Symbols used were Y294[Ref] (▾); Y294[SFI] (▴); Y294[EGI] (♦); Y294[CEL5] glucose preculture (); and Y294[CEL5] PASC preculture (▪).

### DETAILED DESCRIPTION OF THE INVENTION

One aspect of the present invention relates to host cells containing recombinant enzymes which enable the host cells to derive fermentable sugars from complex polysaccharides found in biomass. Specifically, embodiments of the invention enable digestion and utilization of cellulosic material for fermentation by microorganisms. In one embodiment metabolically engineered host cells capable of new biochemical processes that enable growth on cellulosic material as well as concomitant ethanol production are described. The embodiments of the invention disclosed herein provide an important step in effectively using biomass material to produce ethanol, which can be subsequently used for a variety of energy needs.

### DEFINITIONS

A “recombinant host cell” as defined herein is a cell which has one or more exogenous genes in the nucleus of the cell. The exogenous gene(s) can be carried on a plasmid(s) or integrated into the chromosome(s) of the host cell.

“Endoglucanase” as defined herein is an enzyme capable of cutting at random within the polysaccharide chain of cellulose to yield predominantly oligosaccharides and is characterized by EC 3.2.1.4 activity.

“β-glucosidase” as defined herein is an enzyme capable of hydrolyzing soluble cellodextrins and cellobiose to glucose units and is characterized by the EC 3.2.1.21 activity.

“Amorphous cellulose” is defined herein as a cellulosic fraction that is not in compact crystal structure and can absorb water.

One aspect of the present invention relates to a recombinant host cell comprising a heterologous polynucleotide which encodes an endoglucanase and a heterologous polynucleotide which encodes a β-glucosidase, wherein the host cell does not express an exoglucanase, and wherein the host cell can grow on amorphous cellulose as the sole carbon source.

Another aspect of the present invention relates to a recombinant host cell comprising a heterologous polynucleotide which encodes an endoglucanase and a heterologous polynucleotide which encodes a β-glucosidase, wherein the host cell can grow on amorphous cellulose as the sole carbon source, and wherein the host cell does not require pre-growth on a non-amorphous cellulose carbon source.

In one embodiment of the present invention, the recombinant host cell is a yeast. In some embodiments, the recombinant host cell is of the genus Saccharomyces, Kluyveromyces, Candida, Pichia, Schizosaccharomyces, Hansenula, Kloeckera, Schwanniomyces or Yarrowia.

In other embodiments, host cells are yeast of the species S. cerevisiae, S. bulderi, S. barnetti, S. exiguus, S. uvarum, S. diastaticus, K. lactis, K. marxianus or K. fragilis.

The recombinant yeast strains of some aspects of the invention can additionally express other enzymes important for production of ethanol from biomass-derived substrates. Such additional enzymes can be stress-resistance enzymes, such as heat shock proteins, catalases, superoxide dismutases, glutathione reductases and the like. Additionally, other enzymes expressed or over-expressed by the host cells in certain aspects of the invention can be enzymes that increase metabolic flux through various metabolic pathways (including the pentose phosphate pathway), alter glucose repression characteristics of the cell, break down or export fermentation inhibitors from the cell, alter the NAD⁺/NADP⁺ ratio, alter the NAD⁺/NADH ratio, disrupt the respiratory capacity of the cell, increase growth rate, increase fermentation rate, and/or increase ethanol yield.

The recombinant yeast strain of certain aspects of the invention can also contain a deletion or disruption of a native enzyme or enzymes in order to increase ethanol yield directly or indirectly. Examples of enzymes it can be desirable to disrupt or delete in cells in certain aspects of the invention include enzymes which repress the stress response of the cell, alter the flux of carbons away from ethanol production (and thus produce byproducts), allow mating type switching, slow growth, or increase respiratory capacity. Additionally, deletion of enzymatic activities can be useful for creating additional auxotrophic markers which would facilitate the transformation of host cells of certain aspects of the invention.

Additionally, cells of certain embodiments can express heterologous enzymes which allow fermentation of pentose sugars or other carbon containing substrates which can be found in the prefermentation biomass of certain aspects of the invention.

In some embodiments, cells of the present invention can be selected through repeated rounds of adaptation experiments to yield more hearty strains suitable for industrial fermentation, and/or cells can be repeatedly crossed to (by mating) cells pre-selected for good utility in industrial application. Progeny cells can then be selected, according to some aspects of the present invention for ability to ferment amorphous cellulose, and further subjected to mating and/or selection to yield increasingly industrially useful progeny. Methods for increasing the “toughness” of a strain and otherwise maximizing its utility for industrial application will be readily apparent to one of skill in the art.

In some embodiments of the present invention, the endoglucanase and β-glucosidase can be any suitable endoglucanase derived from, for example, a fungal or bacterial source. For example, the endoglucanase can be derived from Holomastigotoides mirabile (e.g. HmEGJ, HmEG2 or HmEG3), Humicola grisea (e.g. EG1), Hypocrea pseudokoningii (e.g. EG1), Aspergillus aculeatus (e.g. EG1), Aspergillus kawachii (e.g. cel5A or cel5B), Aspergillus niger (e.g. engl), Phanerochaete chrysosporium (e.g. Cel5A), or Trichoderma reesei (e.g. EG1) and the β-glucosidase can be derived from, for example, Humicola grisea, Hypocrea pseudokoningii, Aspergillus aculeatus (e.g. bgl1), Aspergillus kawachi (e.g. bglA), Aspergillus nidulans, Aspergillus niger (e.g. bgl1), Hypocrea jecorina (e.g. cel3A), Phanerochaete chrysosporium, Saccharomycopsis fibuligera (e.g. bgl1), or Penicillium brasilianum.

In certain embodiments of the invention, the endoglucanase(s) can be an endoglucanase I or an endoglucanase II isoform, paralogue or orthologue.

In another embodiment, the endoglucanase expressed by the host cells of the present invention can be recombinant endo-1,4-β-glucanase.

In some embodiments of the present invention the endoglucanase is an endo-1,4-β-glucanase from Trichoderma reesei.

In certain embodiments of the present invention the β-glucosidase is derived from Saccharomycopsis fibuligera.

In some embodiments, the β-glucosidase is a β-glucosidase I or a β-glucosidase II isoform, paralogue or orthologue.

In other embodiments, the enzymes expressed by the cells of the present invention can be recombinant endo-1,4-β-glucanase from Trichoderma reesei (teleomorph Hypocrea jadinii) and β-glucosidase from Saccharomycopsis fibuligera source.

In some embodiments, the enzymes can be recombinant endo-1,4-β-glucanase I (EGI—Genbank accession number 1302152A) from Trichoderma reesei (teleomorph Hypocrea jecorina) and β-glucosidase I (BGLI—Genbank accession number P22506) from a Saccharomycopsis fibuligera source.

In some embodiments, the recombinant endo-1,4-β-glucanase I (EGI) from T. reesei and β-glucosidase I (BGLI) from Saccharomycopsis fibuligera can be recombinantly introduced into an industrial strain of Saccharomyces cerevisiae or another yeast suitable for commercial production of ethanol. In one embodiment, the recombinant endo-1,4-β-glucanase I (EGI) from T. reesei and β-glucosidase I (BGLI) from Saccharomycopsis fibuligera are recombinantly produced by Saccharomyces cerevisiae Y294[CEL5].

In some embodiments, the recombinant enzymes of the present invention can be encoded on a common plasmid or by separate plasmids. The plasmids can be high copy plasmids or the more stable lower copy number CEN plasmids. The recombinant enzymes of the present invention can be under the control of any of a variety of promoters suitable for their expression including constitutively active promotors or promotors which can be regulated by conditional variables. In some embodiments, the recombinant enzymes of the present invention can be integrated in to the genomic DNA of the host cell under the control of any suitable promoter.

Additionally, in certain aspects of the invention the recombinant enzymes can be secreted into the extracellular environment, compartmentalized to various organelles, or tethered to the cell membrane or cell wall.

Alternatively, in some embodiments the recombinant enzymes can be expressed as fusion proteins. The enzymes can be fused to each other directly, or separated by a flexible linker region of amino acids. The enzymes of some embodiments of the invention can also be fused to other proteins to promote their secretion, secure them to the cell wall or cell membrane, alter their stability, alter their enzyme kinetics, and/or alter their substrate specificity. Additionally, in some embodiments the enzymes of the present invention can have alternative secretion sequences to affect their secretion kinetics.

An industrial strain of yeast according to the present invention would also be capable of fermentation of native cellulosics if an exogenous cellulase, notably cellobiohydrolase and/or exoglucanase enzymes, is added to the fermentation broth. In one embodiment, a recombinant host cell according to the present invention would be useful in combination with other microbes to ferment various substrates found in biomass to ethanol. This embodiment of the invention enables fermenting native cellulosics that are present in all plant biomass to ethanol. Native cellulosics include both amorphous and crystalline cellulose moieties. As such, one aspect of the invention can include the step of hydrolyzing the amorphous and crystalline cellulosics with the addition of cellulase enzymes.

Another aspect of the invention relates to methods of fermenting amorphous cellulosics which includes the step of hydrolyzing cellulosics by recombinant enzymes produced by the recombiant host cells described herein. In one embodiment, there is provided a method of fermenting amorphous cellulosics to ethanol which includes the steps of hydrolyzing the cellulose chains with endoglucanase and β-glucosidase produced by a recombinant yeast strain and subsequently recovering the ethanol.

In one embodiment, the method of fermenting amorphous cellulosics includes the steps of hydrolyzing the cellulose chains with endoglucanase and β-glucosidase co-produced by a single recombinant yeast strain. In another embodiment, the enzymes are produced by different strains.

In another embodiment of the invention, the endoglucanase and β-glucosidase are secreted and can act on amorphose cellulose to yield simple sugars in the reaction medium. The presence of these sugars can be detected by standard assays.

In some embodiments of the invention, the cellulosics described herein can be pretreated in a variety of ways known to those skilled in the art to generate amorphous cellulosics susceptible to enzyme hydrolysis by recombinant endoglucanase and β-glucosidase produced by host cells of the present invention.

More specifically, cellulosics in compositions of some aspects of the present invention can be pretreated by steam explosion, ammonium explosion treatment, CO₂ explosion, acid or alkali pretreatment, oxidative pretreatment, organosolvent treatment or enzymatic (cellobiohydrolase) treatment to yield amorphous cellulosics.

Compositions comprising amorphous cellulose together with a host cell capable of converting amorphous cellulose into ethanol via fermentation are also provided. Such compositions of the invention can contain amorphous cellulose as the sole carbon source, but alternatively the composition can be supplemented with alternative carbon sources.

In other embodiments, the composition of amorphous cellulose and host cells can contain crystalline cellulose as well as additional nutrients suitable for industrial fermentation including, for example, antibiotics, vitamins, minerals, amino acids, salts, pH modifiers, and other ingredients which will be readily apparent to one of skill in the art.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following example, which is included herewith for purposes of illustration only and is not intended to be limiting of the invention.

### EXAMPLE

### Chemical Components, Media, and Culture Conditions

All chemicals, media components and supplements were of laboratory grade standard. Phosphoric acid swollen cellulose (PASC) was prepared as described by Zhang, Y. H. and Lynd, L. R., Biomacromolecules. 6, 1510-1515 (2005) using Avicel PH-101 (Fluka). E. coli strain XL1 Blue MRF (Stratagene) was used for plasmid transformation and propagation. Cells were grown in LB medium (5 g·L⁻¹ yeast extract, 10 g·L⁻¹ NaCl, 10 g·L⁻¹ tryptone) supplemented with ampicillin (100 mg·L⁻¹). S. cerevisiae Y294 transformants were selected and maintained on SC^{-URA} or SC^{URA-LEU} medium plates (1.7 g·L⁻¹ yeast nitrogen base w/o amino acids and ammonium sulphate [Difco laboratories, Detroit, Mich., USA], 5 g·L⁻¹ (NH₄)₂SO₄, 20 g·L⁻¹ glucose, 15 g·L⁻¹ agar, and supplemented with amino acids as required). Autoselective S. cerevisiae strains were cultured in YPD medium (10 g·L⁻¹ yeast extract, 20 g·L⁻¹ peptone, 20 g·L⁻¹ glucose) and strains expressing the S. fibuligera BGLI were at times cultured on YPC medium (10 g·L⁻¹ yeast extract, 20 g·L⁻¹ peptone, 20 g·L⁻¹ cellobiose). Strain Y294[CEL5] co-expressing the S. fibuligera BGL1 and the T. reesei EG1 were cultured on YP-PASC medium (10 g/L⁻¹ yeast extract, 20 g·L⁻¹ peptone, 10 g·L⁻¹ PASC).

Yeast strains were routinely cultured in 250 mL Erlenmeyer flasks containing 100 mL medium at 30° C., on a rotary shaker at 100 rpm. For aerobic growth, cultures were grown in 50 mL YP medium containing 10 g·L⁻¹ PASC in baffled 250 mL Erlenmeyer flasks on a rotary shaker at 100 rpm and 30° C. Cultures were inoculated to ˜2×10⁵ cells per mL from overnight cultures. For anaerobic fermentation yeast strains were grown in rubber plugged 100-mL glass serum bottles containing 100 mL YP-PASC medium supplemented with 0.01 g·L⁻¹ ergosterol and 0.42 g·L⁻¹ Tween 80 (Yu, S., et al., Appl. Microbiol. Biotechnol. 44, 314-320 (1995)). Precultures of the strains were grown on YPD medium. A precultures of strain Y294[CEL5] was also grown in 10 g·L⁻¹ PASC. For growth on liquid YP-PASC medium three cultures of each strain were inoculated simultaneously. Samples were periodically taken and yeast cells in the media were counted in triplicate on a haemocytometer to produce population growth measurements.

### Microbial Strains and Plasmids

The genotypes and sources of the yeast and bacterial strains, as well as the plasmids that were constructed and used in this example, are summarized in Table 1.

TABLE 1
Microbial Strains and Plasmids Used.

| Strain/Plasmid^{a} | Genotype | Source/Reference |
|---|---|---|
| Yeast and fungal strains: | | |
| Saccharomyces cerevisiae Y294 | α leu2-3,112 ura3-52 his3 trp1-289 | ATCC 201160 |
| S. cerevisiae Y294:^{a} | | |
| (fur1::LEU2 Yep352) | bla ura3/URA3 | This work |
| (fur1::LEU2 ySFI) | bla ura3/URA3 PGK_{P}-XYNSEC-BGL1- | Van Rooyen et al., |
| | PGK_{T} | 2005 |
| (fur1::LEU2 pAZ40) | bla ura3/URA3 ENO1_{P}-EG1-ENO1_{T} | This work |
| (fur1::LEU2 pCEL5) | bla ura3/URA3 PGK_{P}-XYNSEC-BGL1- | This work |
| | PGK_{T} | |
| | ENO1_{P}-EG1-ENO1_{T} | |
| Bacterial strains: | | |
| Escherichia coli | Δ (mcrA)183 Δ(mcrCB-hsdSMR-mrr)173 | ZAP-cDNA Synthesis |
| XL1-Blue MRF′ | endA1 supE44 thi-1 recA1 gyrA96 relA1 | Kit Stratagene |
| | lac[F proAB lacI^{q}ZΔM15 Tn10 (Tet^{I})] | |
| Plasmids: | | |
| YEp352 | bla URA3 | Broach et al., 1979 |
| ySFI | bla URA3 PGKp-XYNSEC-BGL1-PGK_{T} | Van Rooyen et al., |
| | | 2005 |
| pGT1-eg1 | bla gpd_{P}-eg1-glaA_{T} | Rose and Van Zyl, |
| | | 2002 |
| yENO1 | bla URA3 ENO1_{PT} | This work |
| pAZ40 | bla ura3/URA3 ENO1_{P}-EG1-ENO1_{T} | This work |
| pCEL5 | bla ura3/URA3 PGK_{P}-XYNSEC-BGL1- | This work |
| | PGK_{T} | |
| | ENO1_{P}-EG1-ENO1_{T} | |
| pDF1 | bla fur1::LEU2 | La Grange et al., 1996 |

^{a}S. cerevisiae Y294 (fur1::LEU2 YEp352), the reference strain, was designated Y294[REF] S. cerevisiae Y294 (fur1::LEU2 ySFI) was designated Y294[SFI] S. cerevisiae Y294 (fur1::LEU2 pAZ40) was designated Y294[EG1] S. cerevisiae Y294 (fur1::LEU2 yCEL5) was designated Y294[CEL5]

### Plasmid Construction, Cell Transformation, and Growth on Amorphous Cellulose

Standard protocols were followed for DNA manipulations (Sambrook, J., et al., Molecular cloning: a laboratory manual. Cold Spring Harbor Laboratory, Cold Spring Harbor, N.Y. (1989)). Restriction endonuclease-digested DNA was eluted from agarose gels by the method of Tautz and Renz (Anal. Biochem. 132, 503-517 (1983)). Restriction endonucleases, T4 DNA ligase and the Klenow fragment of E. coli DNA polymerase I, were purchased from Roche Molecular Biochemicals and used as recommended by the manufacturer.

For polymerase chain reactions (PCR), Pfu DNA polymerase was purchased from Promega and used as recommended by the manufacturer with a Perkin Elmer GeneAmp® PCR System 2400 (The Perkin-Elmer Corporation, Norwalk, Conn., USA). Details of the primers used in aspects of the present invention are given in Table 2.

TABLE 2
PCR Primers Used for Gene
Isolation and Plasmid Construction.

| | Sequence (5′ → 3′) | Source |
|---|---|---|
| Primer | Restriction sites | accession |
| name | are bold/underlined | nr. |
| ENO1PT | | X99228 |
| ENO1-L | GGATCCACTAGTCTT | |
| | CTAGGCGGGTTATC | |
| ENO1-M | CTAGAAGGCTTAATCA | |
| | AAAGCTCTCGAGATCT | |
| | CGCGAATTCTTTGAT | |
| | TTAGTGTTTGTGTG | |
| ENO1-R | GGATCCAAGCTTGCGG | |
| | CCGCAAAGAGGTTTAG | |
| | ACATTGG | |
| EG1 | | AB003694 |
| TREG1-Left | GATCGAATTCAATGGC | |
| | GCCCTCAGTTACAC | |
| TREG1-Right | GTACAGATCTAGTCAA | |
| | CGCTCTAAAGGCATTG | |
| FUR1 disruption | | M36485 |
| FUR1-L | TCCGTCTGGCATATCCTA | |
| FUR1-R | TTGGCTAGAGGACATGTA | |

The construction of the S. fibuligera BGL1 (SEQ ID NO:3) expressing yeast vector ySFI was previously described (Van Rooyen, R., et al., J. Biotech. 120, 284-295 (2005)). yENOI was constructed by replacing the ADH2_{PT} (Alcohol dehydrogenase II promoter and terminator) cassette of pDLG1 (La Grange et al. Appl. Environ. Microbiol. 62, 1036-1044 (1996) with a 1,030-bp BamHI, Hindle ENO1_{PT} (Enolase I promoter and terminator) overlap PCR fragment (PCR primers ENOL-L, ENO1-M and ENOL-R). The 1,400-bp T. reesei β-1,4-endoglucanase encoding gene (EG1) (SEQ ID NO:1) that was previously cloned (Rose, S. H. and Van Zyl, W. H., Appl. Micobiol. Biotechnol. 58, 461-468 (2002)) was amplified with primers TREG1-Left and TREG1-Right from pGT1-egl and cloned into the EcoRI and BGLII sites of yENO1 under control of the ENO1 promoter and terminator to create pAZ40. The 2,400-bp ENO1_{PT}-EG1 gene cassette was amplified from pAZ40 with the primers ENO1-L and ENO1-R and cloned into the unique BamHI site of ySFI to create pCEL5.

Sequence verification was done after sequence determination by the dideoxy chain termination method, using an ABI PRISM 3100 Genetic Analyzer. Sequence analysis utilized mainly the PC based DNAMAN (version 4.1) package from Lynnon BioSoft and the BLAST program at the National Center for Biotechnology Information (www.ncbi.nih.gov/BLAST/).

DNA transformation of S. cerevisiae Y294 was performed with the lithium acetate dimethylsulfoxide (DMSO) method described by Hill, J., et al., Nucleic Acid Res. 19, 5791 (1991). Autoselective strains were constructed by subsequent transformation with pDF1 (La Grange, D. C., et al., Appl. Environ. Microbiol. 62, 1036-1044 (1996)), to ensure maintenance of the URA3-bearing expression vector under non-selective conditions (Kern, L., et al., Gene 88, 149-157 (1990)); La Grange, D. C., et al., Appl. Environ. Microbiol. 62, 1036-1044 (1996)).

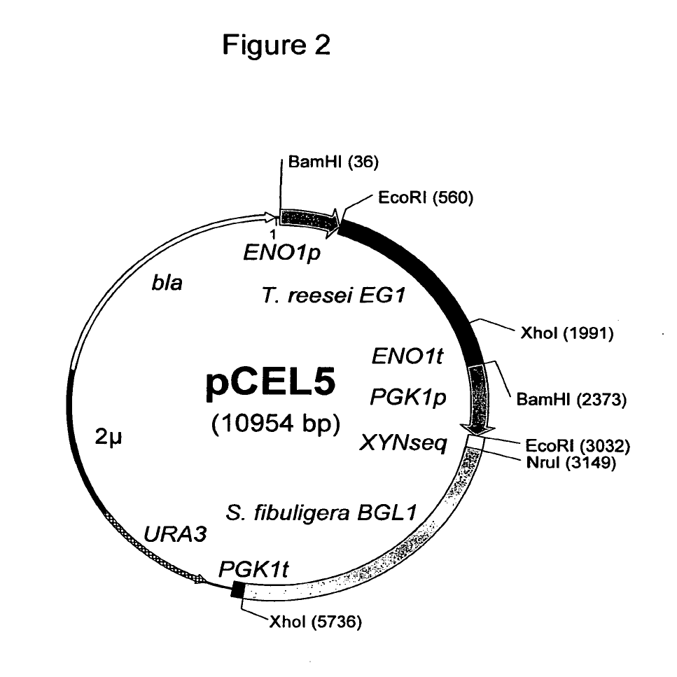

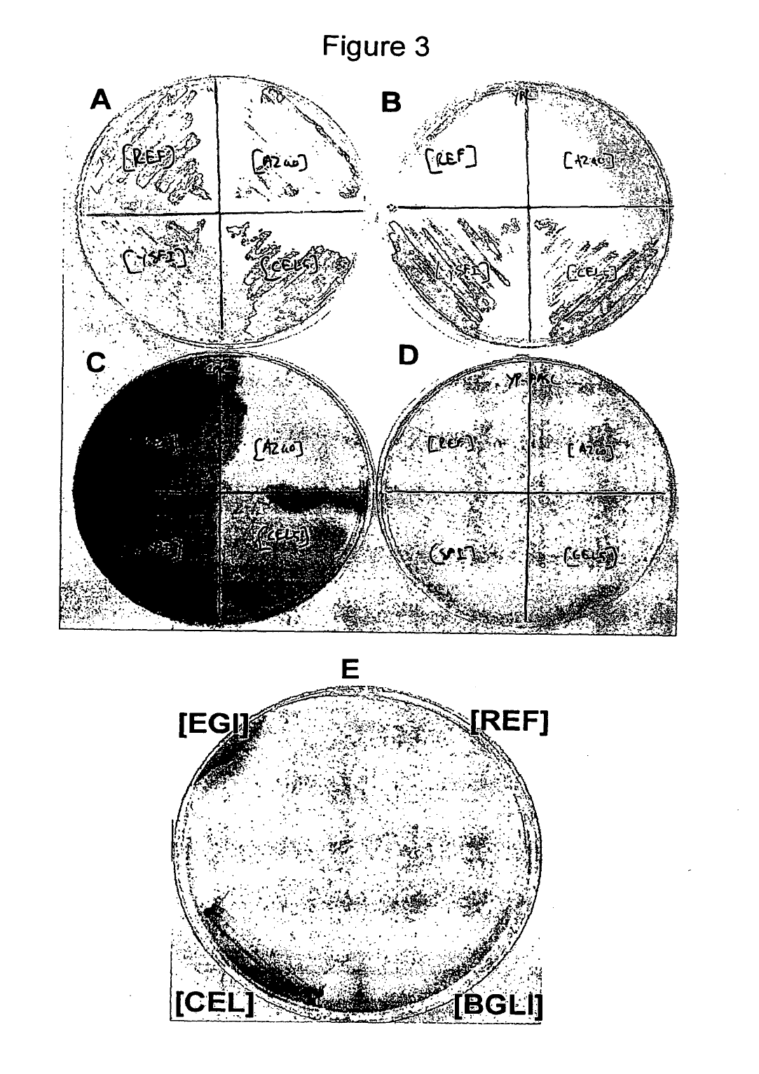

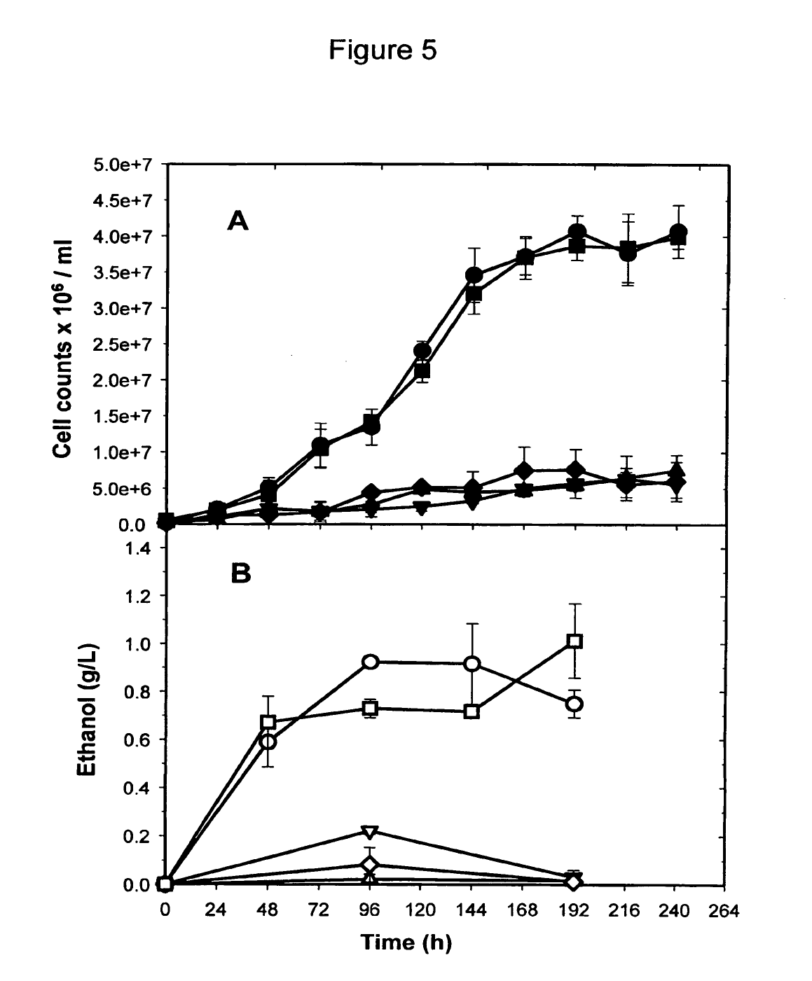

Recombinant yeast strains were constructed that expressed the gene encoding the Saccharomycopsis fibuligera β-glucosidase (BGLI) (SEQ ID NO:3) alone (Y294[SFI]), the gene encoding the Trichoderma reesei endoglucanase 1 (EG1) (SEQ ID NO:1) alone (Y294[EG1]) and a combination of the two genes (Y294[CEL5]—FIG. 2). Heterologous expression of the BGL1 gene enabled the recombinant strains Y294[SFI] and Y294[CEL5] to grow on cellobiose as sole carbohydrate source (FIG. 3B). Heterologous expression of the EG1 gene enabled the recombinant strains Y294[EG1] and Y294[CEL5] to degrade CMC (FIG. 3C). Co-expression of the two cellulase genes enabled the Y294[CEL5] strain to grow on YP-PASC (FIGS. 3D, 5).

### Medium Rheology

Viscosity measurements were done on a Physica MCR 501 (Anton Paar, Germany) using the double gap configuration and heating to 30° C. with a Peltier system (C-PTD200). Flow curves were analysed using the Rheoplus software and were done in three intervals. Interval 1 was a pre-shear on the samples at 2 points (30 s each, shear rate: 1 s⁻¹), interval 2 represented a waiting time before the measurements (5 points, 5 s each) and interval 3 was the analysis phase done over 30 points (5 s each, shear rate 2-200 s⁻¹).

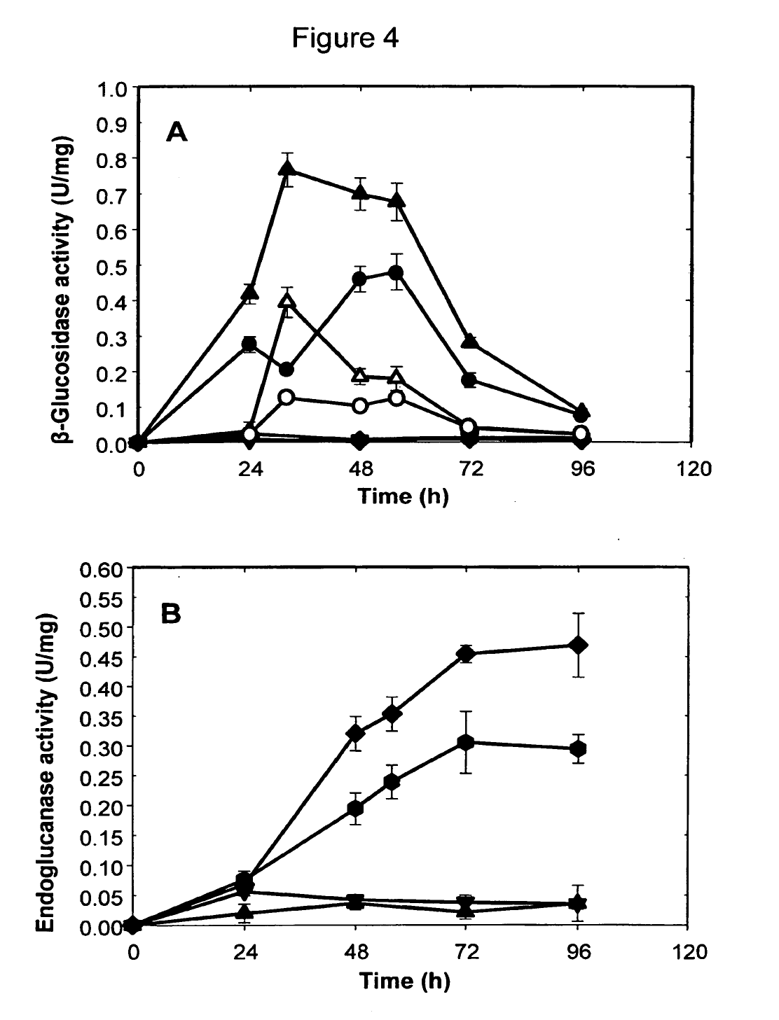

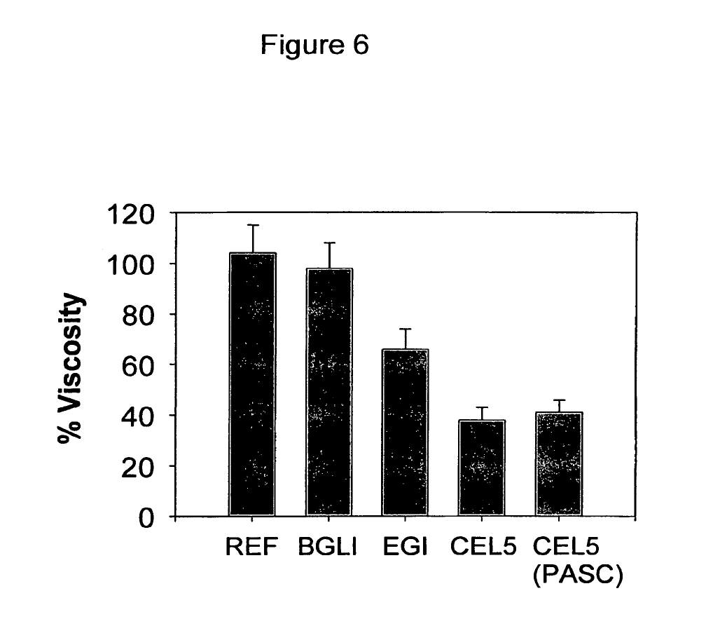

The strains Y294[REF] and Y294[SFI] did not decrease the viscosity of the medium as these strains do not produce endoglucanase activity (FIG. 4). The strain Y294[EG1] lead to some decrease in the viscosity level of the media as it produced endoglucanase activity even though there was little growth of this strain on YP-PASC. The Y294[CEL5] strains led to an almost 60% decrease in medium viscosity reflecting PASC degradation and utilization by this strain. The overall viscosity of the PASC-containing growth media was altered in recombinant cellulase expressing yeast strains (FIG. 6, Table 3).

TABLE 3
Summary of the reduction if medium viscosity due to PASC
degradation, PASC remaining, sugars utilized and ethanol produced
by different recombinant Y294 strains under anaerobic fermentations.

| S. cerevisiae | Relative | Amorphous | Calculated | Ethanol |
|---|---|---|---|---|
| strain | viscosity | cellulose left | Sugars utilized | produced |
| Y294 | (%) | (g/L) | (g/L) | (g/L) |
| [REF] | 103.91 | 9.70 | 0.30 | 0.03 ± 0.03 |
| [SFI] | 98.39 | 9.57 | 0.43 | 0.16 ± 0.01 |
| [EGI] | 66.32 | 8.69 | 1.31 | 0.01 ± 0.03 |
| [CEL5]-G* | 38.29 | 7.47 | 2.53 | 0.75 ± 0.06 |
| [CEL5]-C* | 40.65 | 7.60 | 2.40 | 1.01 ± 0.15 |

*[CEL5]-G-Y294[CEL5] pregrown in YPD medium. [CEL5]-C-Y294[CEL5] pregrown in YP medium containing 10 g/L−¹ PASC.

### Enzymatic Assays

Yeast transformants containing the T. reesei EG1 were screened for carboxymethyl-cellulose (CMC) degrading ability by patching on SC^{-URA} medium containing 0.1% (w/v) CMC (Sigma) with 20 g·L⁻¹ glucose as carbon source. After 48 h of growth, colonies were washed of the plate and the remaining CMC was stained with 0.1% Congo red and de-stained with 1% (w/v) NaCl. Extracellular endoglucanase activity was indicated by clearing zone formation. Endoglucanase activity was quantified as described by Bailey, M. J., et al., J. Biotechnol. 23, 257-270 (1992), in citrate buffer (0.05 M, pH 5, 50° C.) with 1% CMC as substrate. β-Glucosidase activity was measured by incubating appropriately diluted cells or supernatant with 5 mM of p-nitrophenyl-β-D-glucopyranoside (pNPG) in citrate buffer (0.05 M, pH 5.4, 55° C.) for 2 min (Van Rooyen, R., et al., J. Biotech. 120, 284-295 (2005)). The p-nitrophenol released from pNPG was detected at 405 nm after adding 1 mL of 1 M Na₂CO₃ to raise the pH and stop the reaction. All enzymatic assays were done in triplicate from 100 mL YPD cultures (3 per strain) and expressed in units per mg dry cell weight (Meinander, N., et al., Microbiology 142, 165-172 (1996)) where one unit was defined as the amount of enzyme required to produce 1 μmol of p-nitrophenol or reducing sugar per minute under the assay conditions.

Heterologous enzyme production was quantified (FIG. 4) and it was shown that β-glucosidase activity was mostly cell associated despite the fact that a secretion signal (T. reesei xyn2 secretion signal) preceded the BGL1 gene. Co-expression of the β-glucosidase and endoglucanase genes led to slightly lower levels of activity when compared to individual expression.

### Component Small Molecule Analysis

Cellobiose, glucose, glycerol, acetate and ethanol concentrations were determined by high performance liquid chromatography (HPLC), with a Waters 717 injector (Milford, Mass., USA) and Agilent 1100 pump (Palo Alto, Calif., USA). The compounds were separated on an Aminex HPX-87H column (Bio-Rad, Richmond, Calif.), at a column temperature of 45° C. with 5 mM H₂SO₄ as mobile phase at a flow rate of 0.6 mL·min⁻¹ and subsequently detected with a Waters 410 refractive index detector.

Growth in liquid YP medium containing 10 g·L⁻¹ PASC as sole carbohydrate source led to concomitant ethanol production (FIG. 5, Table 3). Anaerobic growth yielded 3.88×10⁷ cells ml⁻¹ or approximately 0.27 dry cell weight (DCW) and 1.0 g·L⁻¹ ethanol. As anaerobic DCW yields are ˜0.1 g/g glucose (Van Dijken, J. P., et al., Enzyme Microb. Technol. 26, 706-714 (2000)), it follows that ˜2.7 g·L⁻¹ glucose or 27% of the PASC was obtained through enzymatic conversion. As the theoretical optimum ethanol yield from glucose is 0.51 g·g⁻¹ a maximum of 1.367 ethanol from 2.7 g·L⁻¹ glucose could be expected. The yield of ˜1 g·L⁻¹ was thus 73% of the theoretical maximum

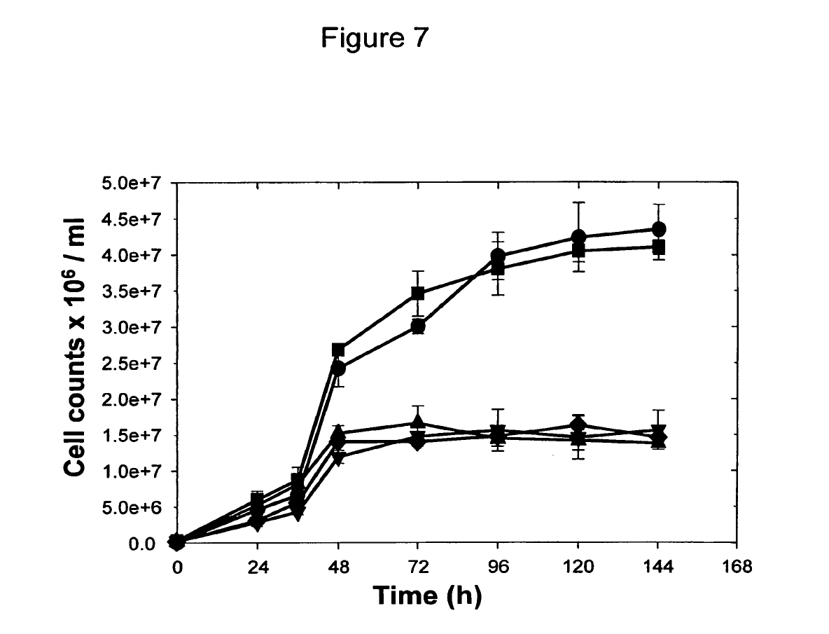

Aerobic cultivations yielded cell counts similar to those found for anaerobic cultivations but in a considerably shorter timeframe (FIG. 7). It was previously shown that in aerobic cultivations, S. cerevisiae biomass accumulates to approximately 5 fold the amount that it does in anaerobic cultivations on the same amount of sugar (Van Dijken, J. P., et al., Enzyme Microb. Technol. 26, 706-714 (2000)).

The entire disclosure of all publications (including patents, patent applications, journal articles, laboratory manuals, books, or other documents) cited herein are hereby incorporated by reference.

1. A recombinant host cell comprising:
(a) a heterologous polynucleotide which encodes an endoglucanase; and
(b) a heterologous polynucleotide which encodes a β-glucosidase,
wherein said host cell does not express an exoglucanase, and wherein said host cell can grow on amorphous cellulose as the sole carbon source.

2. A recombinant host cell comprising:
(a) a heterologous polynucleotide which encodes an endoglucanase; and
(b) a heterologous polynucleotide which encodes a β-glucosidase,
wherein said host cell can grow on amorphous cellulose as the sole carbon source, and wherein said host cell does not require pre-growth on a non-amorphous cellulose carbon source.

3. The recombinant host cell of claim 1 wherein said host cell is a yeast.

4. The recombinant host cell of claim 3 wherein said host cell is of the genus Saccharomyces, Kluyveromyces, Candida, Pichia, Schizosaccharomyces, Hansenula, Kloeckera, Schwanniomyces or Yarrowia.

5. The recombinant host cell of claim 4 wherein said host cell is Saccharomyces cerevisiae.

6. The recombinant host cell of claim 1 wherein said endogluconase is derived from Holomastigotoides mirabile, Humicola grisea, Hypocrea pseudokoningii, Aspergillus aculeatus, Aspergillus kawachii, Aspergillus niger, Phanerochaete chrysosporium, or Trichoderma reesei.

7. The recombinant host cell of claim 6 wherein said endogluconase is derived from Trichoderma reesei.

8. The recombinant host cell of claim 1 wherein said endogluconase is an endo-1,4-β-glucanase.

9. The recombinant host cell of claim 8 wherein said endogluconase is an endo-1,4-β-glucanase derived from Trichoderma reesei.

10. The recombinant host cell of claim 1 wherein said β-glucosidase is derived from Humicola grisea, Hypocrea pseudokoningii, Aspergillus aculeatus, Aspergillus kawachii, Aspergillus niger, Phanerochaete chrysosporium, or Saccharomycopsis fibuligera.

11. The recombinant host cell of claim 10 wherein said β-glucosidase is derived from Saccharomycopsis fibuligera.

12. The recombinant host cell of claim 1 wherein said β-glucosidase is a β-glucosidase I.

13. The recombinant host cell of claim 12 wherein said β-glucosidase is a β-glucosidase I derived from Saccharomycopsis fibuligera.

14. The recombinant host cell of claim 1 wherein said endogluconase is an endo-1,4-β-glucanase derived from Trichoderma reesei and said p-glucosidase is a β-glucosidase I derived from Saccharomycopsis fibuligera.

15. The recombinant host cell of claim 1 wherein the recombinant host cell can produce ethanol using amorphous cellulose as a carbon source.

16. The recombinant host cell of claim 1 wherein said endoglucanase and said β-glucosidase are secreted.

17. A method of producing ethanol comprising:
(a) contacting a composition comprising amorphous cellulose with a recombinant host cell of claim 1 wherein said host cell ferments amorphous cellulose to ethanol; and
(b) recovering the ethanol.

18. The method of producing ethanol of claim 17 wherein the composition contains an enzymatically detectable amount of monosaccharide during the fermentation process.

19. The method of claim 18 further comprising contacting said composition with an additional microorganism.

20. The method of producing ethanol of claim 17, wherein said cellulose has been pretreated to yield amorphous cellulose.

21. A composition comprising the host cell of claim 1 and amorphous cellulose.

22. The composition of claim 21 further comprising crystalline cellulose.

23. The composition of claim 22 further comprising a host cell that expresses an exoglucanase.

24. The recombinant host cell of claim 2 wherein said host cell is a yeast.

25. The recombinant host cell of claim 24 wherein said host cell is of the genus Saccharomyces, Kluyveromyces, Candida, Pichia, Schizosaccharomyces, Hansenula, Kloeckera, Schwanniomyces or Yarrowia.

26. The recombinant host cell of claim 25 wherein said host cell is Saccharomyces cerevisiae.

27. The recombinant host cell claim 2 wherein said endogluconase is derived from Holomastigotoides mirabile, Humicola grisea, Hypocrea pseudokoningii, Aspergillus aculeatus, Aspergillus kawachii, Aspergillus niger, Phanerochaete chrysosporium, or Trichoderma reesei.

28. The recombinant host cell of claim 27 wherein said endogluconase is derived from Trichoderma reesei.

29. The recombinant host cell of claim 2 wherein said endogluconase is an endo-1,4-β-glucanase.

30. The recombinant host cell of claim 29 wherein said endogluconase is an endo-1,4-β-glucanase derived from Trichoderma reesei.

31. The recombinant host cell of claim 2 wherein said β-glucosidase is derived from Humicola grisea, Hypocrea pseudokoningii, Aspergillus aculeatus, Aspergillus kawachii, Aspergillus niger, Phanerochaete chrysosporium, or Saccharomycopsis fibuligera.

32. The recombinant host cell of claim 31 wherein said β-glucosidase is derived from Saccharomycopsis fibuligera.

33. The recombinant host cell of claim 2 wherein said β-glucosidase is a β-glucosidase I.

34. The recombinant host cell of any claim 33 wherein said β-glucosidase is a β-glucosidase I derived from Saccharomycopsis fibuligera.

35. The recombinant host cell of claim 2 wherein said endogluconase is an endo-1,4-β-glucanase derived from Trichoderma reesei and said β-glucosidase is a β-glucosidase I derived from Saccharomycopsis fibuligera.

36. The recombinant host cell of claim 2 wherein the recombinant host cell can produce ethanol using amorphous cellulose as a carbon source.

37. The recombinant host cell of claim 2 wherein said endoglucanase and said β-glucosidase are secreted.

38. A method of producing ethanol comprising:
(a) contacting a composition comprising amorphous cellulose with a recombinant host cell of claim 2 wherein said host cell ferments amorphous cellulose to ethanol; and
(b) recovering the ethanol.

39. The method of producing ethanol of claim 38 wherein the composition contains an enzymatically detectable amount of monosaccharide during the fermentation process.

40. The method of claim 38 further comprising contacting said composition with an additional microorganism.

41. The method of producing ethanol of claim 38 wherein said cellulose has been pretreated to yield amorphous cellulose.

42. A composition comprising the host cell of claim 2 and amorphous cellulose.

43. The composition of claim 42 further comprising crystalline cellulose.

44. The composition of claim 43 further comprising a host cell that expresses an exoglucanase.
